(19) United States
(12) Patent Application Publication
(10) Pub. No.: US 2007/0122306 A1
(43) Pub. Date: May 31, 2007

(54) Device

(75) Inventors: Colin Brown, Berkshire (GB); Guy Edward Naish, Oxfordshire (GB); Kishen Gohil, Surrey (GB)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 10/572,823

(22) PCT Filed: Sep. 23, 2004
(86) PCT No.: PCT/CH04/00599

§ 371 Date: Sep. 22, 2006

(30) Foreign Application Priority Data
Sep. 26, 2003 (GB) 0322566.1

(51) Int. Cl. A61L 9/12

(52) U.S. Cl. 422/5; 422/124

(57) ABSTRACT

A device adapted to disseminate vaporous material, such as a fragrance, into an atmosphere by means of forced ventilation acting on an evaporation surface (6) supplied with a liquid volatile material that is vaporised thereby, the evaporation surface being essentially planar and having an orientation generally parallel to the direction of the forced ventilation. The ventilation is preferably supplied by an electrically-powered fan (8).

This invention relates to a device for disseminating vaporous material in an enclosed atmosphere by means of forced ventilation.

Devices for disseminating vaporous materials in an enclosed atmosphere, such as a room or hall, are very well known, and many varieties are available. The vaporous material is generally provided in the form of a volatile liquid comprising a substance whose presence in the atmosphere is desired, for example, a fragrance or an insecticide. Most of the known devices for home and non-institutional use are simple and inexpensive, which has led to their wide acceptance. A high proportion of these works by evaporation alone, typically from a cylindrical porous wick that extends from the liquid in a reservoir to the atmosphere. This is often not sufficient, and assistance with evaporation has been introduced. This has typically taken the form of some type of heating element used in conjunction with an evaporating element. Such a device can work well, but it has its drawbacks.

Forced ventilation by means of some sort of artificially-created air current has long been an attractive idea, but until very recently expense has limited its use to large installations; it has been prohibitively expensive for small devices of the type used in homes and other relatively small enclosed atmospheres. This situation has changed with the widespread availability of small electric cooling fans, designed initially for the computer industry. This has made possible the production of cheap, fan-driven (battery or mains) devices for the dissemination of vaporous materials, and commercial examples have started to appear. Present designs feature the use of such fans with conventional cylindrical wicks, which protrude into the air stream created by the fan.

It has now been found that such devices can be substantially improved in performance while keeping the prices down. The invention therefore provides a device adapted to disseminate vaporous material into an atmosphere by means of forced ventilation acting on an evaporation surface supplied with a liquid volatile material that is vaporised thereby, the evaporation surface being essentially planar and having an orientation generally parallel to the direction of the forced ventilation.

By “forced ventilation” is meant any form of ventilation in which a flow of ventilating gas is artificially caused to flow in a desired direction. This can be achieved by any convenient means, such as the release of a gas from a pressurised cylinder. However, for the purposes of this invention, it is preferred that the gas be atmospheric air (with relation to the flowing gas, the word “air” will be used exclusively from now on, but it should be remembered that this is considered to encompass other gases). It may be caused to flow by any convenient means, for example, a bellows, but it is preferred that the flow be continuous and substantially uniform. This can be achieved by simple devices such as impellers or fans. These may be driven by any convenient means of motivation, but it is preferred that the means be electricity, provided by, for example, mains electricity, solar cells and batteries. The nature and desired location of the device will determine which type of electrical generation will be most appropriate (for example, batteries for a device that is required to operate in locations where mains electricity is not easily available). If mains electricity is to be used, the device may be provided with a power cable and plug, or a plug built into the structure of the device, so that it can be directly plugged into a power point. Such a device will also need a transformer, but this is well within the skill of the art.

The significant difference between the present invention and the devices known to the art is that the evaporation surface is essentially planar. In other words, it is not a three-dimensional solid, like a conventional cylindrical wick protruding into the flow of forced ventilation, but a substantial surface across which the air flows. This does not mean that some element of the evaporation surface cannot protrude into the air flow (this aspect will be further discussed hereinunder), but that the basic evaporation surface is planar and lies in a plane generally parallel to the direction of air flow. The surface can be in any orientation around the axis of air flow, but it is preferred for practical reasons that it be generally horizontal and located beneath the flow. This allows for ease of operation and replacement of components and replenishment of liquid.

In its simplest form, the evaporation surface is completely flat and is positioned such that the air flow of the forced ventilation blows across this surface in a direction parallel to the plane of the surface. The surface is preferably appreciably larger than the surface area of the typical cylindrical wick that is impinged upon by the air flow in a known device. In a typical household application, the diameter will be typically of the order of 3-4 cm, as opposed to the wick diameter typically of about 0.5-1.5 cm. (these measurements are given by way of illustration and are not to be regarded as being in any way limiting). Because of its simplicity and cheapness, this embodiment is one of the most preferred embodiments. Thus, in a typical device for domestic use, the evaporative surface may be an insert in the neck of a reservoir of volatile liquid, and the liquid may be brought to the evaporative surface by any convenient means, for example, an attached wick extending downwardly into the liquid. Such reservoirs can be made available as separate units, able to be fitted to a standard forced ventilation unit equipped with a suitable attachment means, for example, a neck bearing an external screw thread that cooperates with a screw thread in the ventilation unit, the screwing in of the reservoir bringing the evaporation surface in the neck into the appropriate evaporative relationship with the air flow. There are many other possibilities, such as a variety of snap fittings, but the nature of the attachment means is not narrowly critical and the skilled person will readily be able to envisage a wide range of possibilities, all within the scope of the invention.

It is also possible that the evaporation surface may not be completely flat. For example, it may comprise undulations of any desired form, for example, concave “wave”-type ridges, or the opposite, convex “bump”-type ridges. The form is not critical. These have the effect of increasing the surface area, and they may protrude into the air flow.

In a particularly preferred embodiment, there is raised on, and essentially perpendicular to, the evaporation surface at least one flat vane, which extends across the surface in the direction of the air flow. Although “vane” is used in the singular here, there may be more than one, and the use of the singular also includes the plural; indeed, there is preferably a series of such vanes, arranged parallel to each other. The vane may extend completely across the surface, or it may be made up of a series of short vanes extending along a common axis parallel to the direction of air flow. The height to which the vane extends from the surface is not narrowly critical, and suitable dimensions can be decided by the skilled person. The vane may protrude into or through the air flow to any desired degree. The vane may be fitted to an evaporative surface, or it may be integral therewith.

In one preferred embodiment, the surface may be provided with a vane adapted to be rotated from a position parallel to the gas flow to a flow-blocking position transverse to the flow. In this case, the vane should extend sufficiently from the evaporation surface to allow blocking of the flow. This is a convenient way of regulating the flow without turning off or unplugging the apparatus. The rotation may be achieved by any convenient means, usually (and preferably) by making the evaporation surface rotatable about an axis transverse to the direction of the gas flow.

Evaporative surfaces may be made from any suitable material, and the skilled person may easily select such a material. The surface, the means of transporting the liquid from the reservoir to the surface and, where present, the vane, may be made as different components from different materials and assembled. Alternatively, depending on the desired materials, two or more of these components may be integral. For example, the surface and the vane may be machined or moulded from a single material, with the transporting means (for example, a wick) being made separately. It is possible to make all three in one piece. Typical materials for use in such surfaces, vanes or wicks are porous plastics and ceramic materials .

A particularly preferred embodiment of the invention comprises the following elements:
(a) an electrically-driven fan, fitted with a housing that is constructed so that it blows a current of air horizontally through an exit port into an atmosphere;
(b) a reservoir of volatile liquid for evaporation into the atmosphere, the reservoir having an upper orifice substantially completely blocked by an essentially planar, essentially horizontal evaporation surface, reservoir and housing cooperating such that the current of air blows across the evaporation surface as it moves towards the exit port; and
(c) means of transferring liquid from the reservoir to the evaporation surface.

By “substantially completely blocked” is meant that the evaporative surface blocks the upper orifice to a sufficient extent such that the liquid will not flow out to any great extent if the reservoir is knocked over, but that there will be provision for pressure to be equalised between the atmosphere and the interior of the reservoir. (If there is no such provision, evaporation can cause a partial vacuum, which will stop the device from working). Suitable air access may be provided by any convenient means, for example, choice of material or the provision of a suitable small vent.

The devices according to the invention are cheap and easy to manufacture and use, and reliable in service. A typical device will have an integral electrical plug and can simply be plugged into a power point and left to operate. They also perform much better than do the known devices. In addition, where replaceable reservoirs of volatile liquid are to be used, these are more easily sealed that those currently used, resulting in less wastage.

The invention also provides a method of disseminating into an atmosphere a volatile liquid whose presence is desired there, comprising
(a) the provision of a gas flow into the atmosphere;
(b) the location of an essentially planar evaporation surface in relation to the gas flow, such that the flow is across the surface and essentially parallel to it; and
(c) the provision to the evaporation surface of a supply of volatile liquid.

The invention shall now be further described with reference to the accompanying drawings, which depict a preferred embodiment particularly adapted for the dissemination of fragrance, and which are not intended to be in any way limiting.

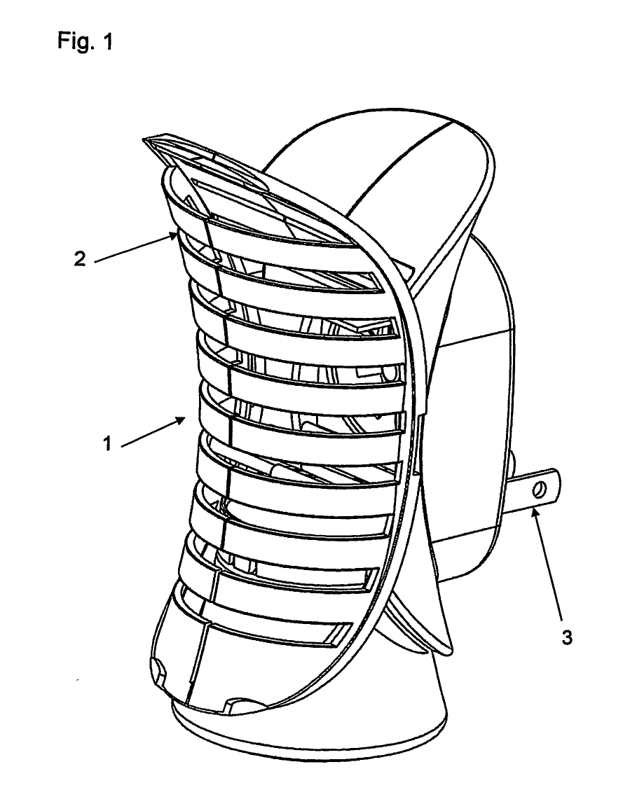

FIG. 1 is a perspective drawing of an embodimnent of the invention, showing the general configuration.

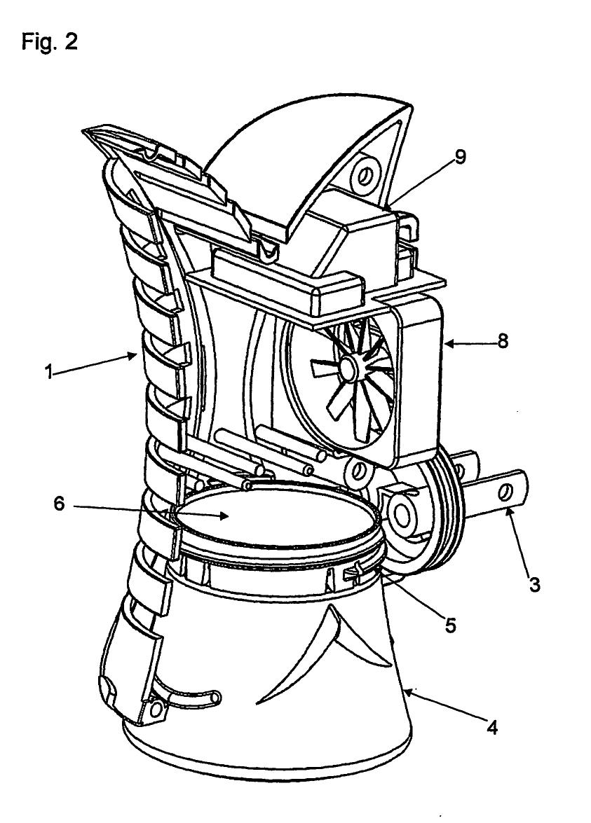

FIG. 2 is a diagrammatic part-cutaway drawing of the embodiment of FIG. 1, showing the positioning of the important elements of the embodiment.

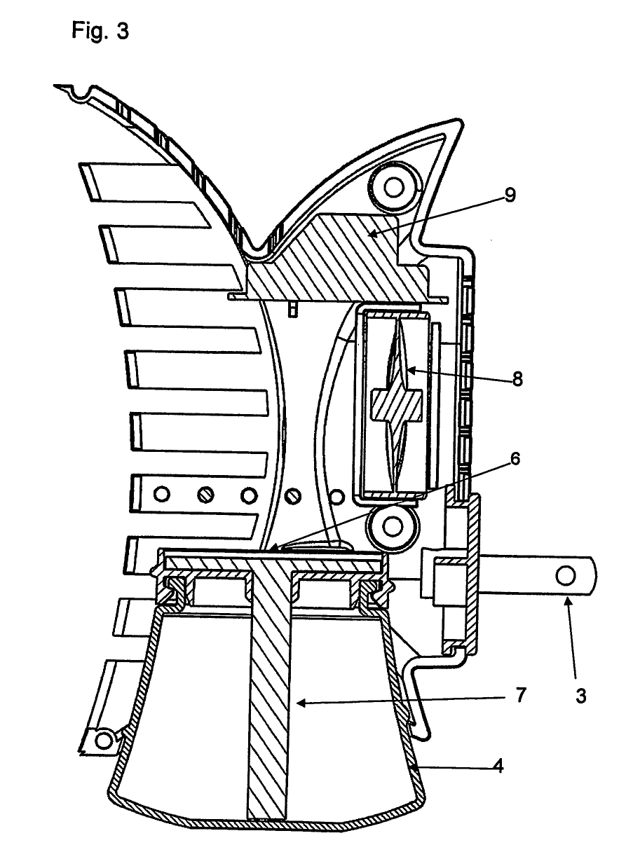

FIG. 3 is a diagrammatic vertical cross-section through the embodiment of FIG. 1.

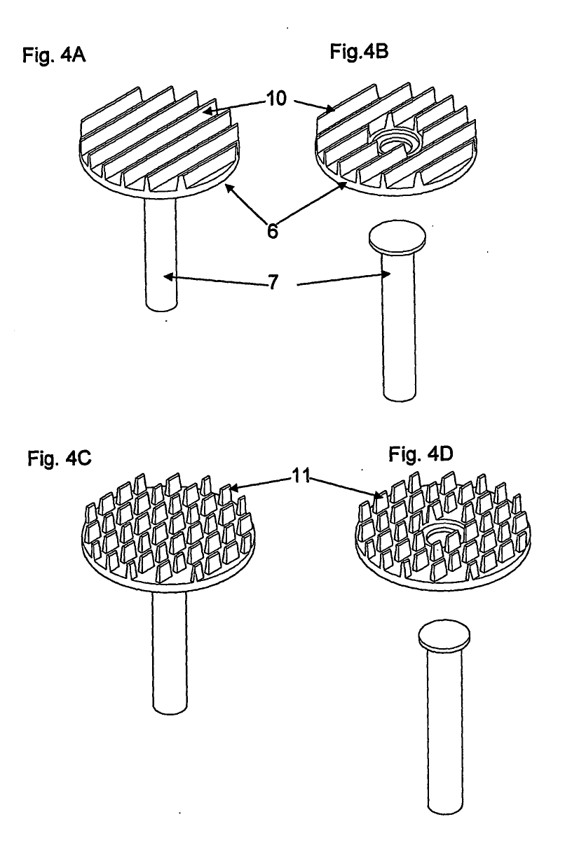

FIG. 4 depicts diagrammatically some possibilities of auxiliary evaporation surfaces.

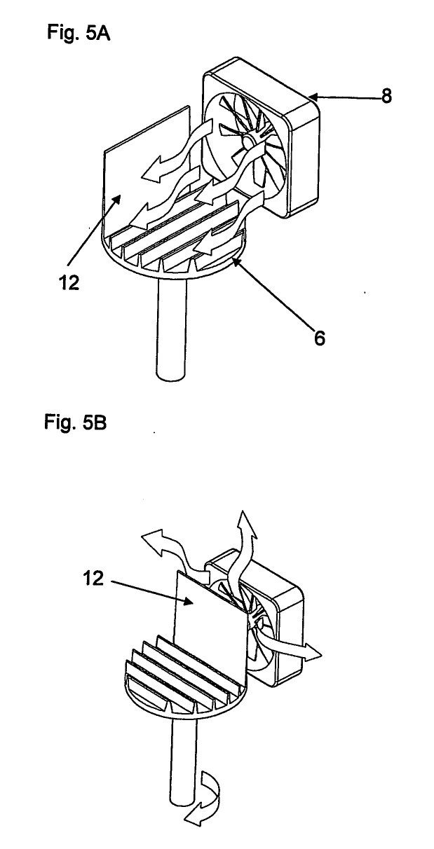

FIG. 5 depicts diagrammatically a further embodiment that makes use of auxiliary evaporation surfaces.

It will be understood that the shape of the device depicted in the figures is largely a matter of aesthetics and that many other shapes are possible. Moreover, it will also be understood that, using the ordinary skill of the art, the internal arrangement and features may be varied considerably from those shown, without departing from the concept of the invention.

The device comprises an outer housing 1 made of plastics or other suitable material. This housing comprises a row of slots 2 for the dissemination of fragrance into the atmosphere. At the rear of the housing are located the prongs of a electrical plug 3, permitting the plugging of the device into a mains power supply.

Located within the housing is a fragrance reservoir 4, this being held in place by means of a screw thread 5 on the neck of the reservoir, which cooperates with a screw thread on the inside of the housing. The neck of the reservoir is wide and is closed by a large evaporating surface 6. This is attached in turn to a porous wick 7, which extends downwards into the reservoir 4 and brings liquid to the evaporating surface 6.

A stream of air is caused to flow across the evaporating surface 6 in the direction of the slots 2 by means of fan 8. The fan is of the type used in laptop computers and is electrically powered by current drawn from a power point by means of the plug 3, the mains voltage being converted to an appropriate voltage for the fan by a transformer 9 mounted above the fan. Thus, the act of plugging the device into a power point starts the operation of the fan.

The reservoir 4 is designed for easy removal and replacement. Thus, an exhausted reservoir is readily replaced by simply unscrewing the exhausted one and replaceing it with a full one.

In FIG. 4, there may be seen some non-limiting possibilities of auxiliary evaporation surfaces. The embodiments FIG. 4A and FIG. 4B depict basically the same embodiment, the difference being that, in FIG. 4A, the wick 7 is moulded in a single piece with the planar evaporation surface 6, whereas in FIG. 4B, it is a separate component. The latter arrangement is more convenient to manufacture. However, the two embodiments function in exactly the same way. The same is true of FIG. 4C and 4D.

In FIG. 4A-4B, there is raised on the planar surface 6 a series of parallel rows of vanes 10, extending substantially perpendicularly from the planar surface. These vanes are arranged such that they are parallel to the direction of a flow of forced ventilation. They raise substantially the surface area available for evaporation.

In FIG. 4C-4D, the vanes are broken into series of individual blocks 11. These provide even more surface area and evaporative potential.

In FIG. 5A, the planar surface 6 has a configuration similar to those of FIG. 4A, except for the presence at one side of the parallel row of vanes 10 of a substantially taller vane 12, this extending away from the surface 6 to a distance as great as the diameter of a ventilation fan 8 place so as to blow air across the planar surface. In this case, the planar surface is rotatable in the plane of the surface.

When the vanes are orientated such that they are parallel to the ventilation flow, the device works as previously described. However, when the surface is turned, the taller vane 12 moves in front of the fan 8, preventing its flow from blowing across the planar surface 6. This reduces considerably the amount of fragrance disseminated into the atmosphere, and it thus allows fragrance output to be reduced, without unplug the device.

1. A device adapted to disseminate vaporous material into an atmosphere by means of forced ventilation acting on an evaporation surface supplied with a liquid volatile material that is vaporised thereby, the evaporation surface being essentially planar and having an orientation generally parallel to the direction of the forced ventilation.

2. A device according to claim 1, in which the evaporation surface is generally horizontal and located beneath the flow of the forced ventilation.

3. A device according to claim 1, in which the surface comprises undulations.

4. A device according to claim 1, in which there is raised on the planar surface at least one flat vane, which is essentially perpendicular to the surface and which extends across the surface in the direction of the air flow.

5. A device adapted to disseminate vaporous material into an atmosphere, comprising the following elements:
(a) an electrically-driven fan, fitted with a housing that is constructed so that the fan blows a current of air horizontally through an exit port into the atmosphere;
(b) a reservoir of volatile liquid for evaporation into the atmosphere, the reservoir having an upper orifice substantially completely blocked by an essentially planar, essentially horizontal evaporation surface, reservoir and housing cooperating such that the current of air blows across the evaporation surface as it moves towards the exit port; and
(c) means for transferring liquid from the reservoir to the evaporation surface.

6. A device according to claim 4, in which at least one vane is adapted to be rotated from a position parallel to the gas flow to a flow-blocking position transverse to the flow.

7. A method of disseminating into an atmosphere a volatile liquid whose presence is desired there, comprising
(a) the provision of a gas flow into the atmosphere;
(b) the location of an essentially planar evaporation surface in relation to the gas flow, such that the flow is across the surface and essentially parallel to it; and
(c) the provision to the evaporation surface of a supply of volatile liquid.
